(19) United States
(12) Patent Application Publication
(10) Pub. No.: US 2020/0268440 A1
(43) Pub. Date: Aug. 27, 2020

(54) AUTOMATED ABLATION CONTROL SYSTEMS

(71) Applicant: Acessa Health Inc., Austin, TX (US)

(72) Inventors: Brian J. BERGERON, Austin, TX (US); Kim BRIDGES RODRIGUEZ, Austin, TX (US)

(73) Assignee: Acessa Health Inc., Austin, TX (US)

(21) Appl. No.: 16/735,341

(22) Filed: Jan. 6, 2020

Related U.S. Application Data
(60) Provisional application No. 62/810,145, filed on Feb. 25, 2019.

(51) Int. Cl. A61B 18/14; A61B 34/37

(52) U.S. Cl. CPC A61B 18/1485; A61B 2018/00577; A61B 34/37

(57) ABSTRACT

Automated ablation control systems are described where the ablation system may generally comprise a surgical workstation having one or more robotic arm assemblies and configured to be in proximity to a surgical region of interest and a control station in communication with the surgical workstation and configured to control a positioning of each of the robotic arm assemblies. The system may also include an ultrasound instrument operably coupled to a first robotic arm assembly, an ablation instrument having a plurality of deployable stylets reconfigurable from a low-profile configuration to a deployed configuration, wherein the ablation instrument is operably coupled to a second robotic arm assembly, and an imaging instrument operably coupled to a third robotic arm assembly.

### CROSS-REFERENCE TO RELATED APPLICATIONS

This application claims priority to U.S. Patent Application No. 62/810,145 filed Feb. 25, 2019, the content of which is incorporated herein by reference in its entirety.

### FIELD OF THE INVENTION

The present invention relates to an ablation system positionable in a patient's body for ablation of a tumor, such as a uterine fibroid. More particularly, the present invention relates to an ablation system which may be controlled and/or assisted by a robotically controlled platform.

### BACKGROUND OF THE INVENTION

Advances in technology have resulted in systems that allow a practitioner or other medical professional to remotely control the operation of a medical device. However, only a relatively small percentage of surgeries currently use minimally invasive techniques due to limitations of minimally invasive surgical instruments and techniques currently used, and the difficulty experienced in performing surgeries using such traditional instruments and techniques.

Advances in minimally invasive surgical technology could dramatically increase the number of ablation surgical procedures performed in a minimally invasive manner. To perform surgical procedures, the surgeon typically passes these working tools or instruments through the cannula sleeves to the internal surgical site and manipulates the instruments or tools from outside the abdomen by sliding them in and out through the cannula sleeves, rotating them in the cannula sleeves, levering (i.e., pivoting) the instruments against the abdominal wall and actuating the end effectors on distal ends of the instruments from outside the abdominal cavity. The instruments normally pivot around centers defined by the incisions which extend through the muscles of the abdominal wall. The surgeon typically monitors the procedure by means of a television monitor which displays an image of the surgical site captured by the laparoscopic camera. Typically, the laparoscopic camera is also introduced through the abdominal wall so as to capture the image of the surgical site.

There are many disadvantages relating to such traditional minimally invasive surgical (MIS) techniques. For example, existing MIS instruments deny the surgeon the flexibility of tool placement found in open surgery. Difficulty is experienced in approaching the surgical site with the instruments through the small incisions. The length and construction of many endoscopic instruments reduces the surgeon's ability to feel forces exerted by tissues and organs on the end effector. Furthermore, coordination of the movement of the end effector of the instrument as viewed in the image on the television monitor with actual end effector movement is particularly difficult, since the movement as perceived in the image normally does not correspond intuitively with the actual end effector movement. Accordingly, lack of intuitive response to surgical instrument movement input is often experienced. Such a lack of intuitiveness, dexterity and sensitivity of endoscopic tools has been found to be an impediment in the expansion of the use of minimally invasive surgery.

Minimally invasive telesurgical systems for use in surgery have been and are still being developed to increase a surgeon's dexterity as well as to permit a surgeon to operate on a patient in an intuitive manner. Telesurgery is a general term for surgical operations using systems where the surgeon uses some form of remote control, e.g., a servomechanism, or the like, to manipulate surgical instrument movements, rather than directly holding and moving the tools by hand. In such a telesurgery system, the surgeon is typically provided with an image of the surgical site on a visual display at a location remote from the patient. The surgeon can typically perform the surgical procedure such as an ablation procedure for treating uterine fibroids at the location remote from the patient while viewing the end effector movement on the visual display during the surgical procedure. The surgeon may perform the surgical procedures on the patient by manipulating master control devices at the remote location, which master control devices control motion of the remotely controlled instruments.

### BRIEF SUMMARY OF THE INVENTION

A system is provided for remote robotic control of an ablation system having an imaging device such as a laparoscope or endoscope which is positionable to image an area being subject to surgery. An ultrasound imaging probe may provide a second image output to image the area being subjected to surgery and an ablation probe having a plurality of deployable stylets may be used to ablate the tissue region of interest, such as uterine fibroids.

Software, resident in a computer may receive information received from the surgical device and the imaging devices for display of a unitary image upon a graphic user interface. The system and methods provides for a surgeon to remotely control a radiofrequency (RF) ablation device, view operating parameters and record information associated with the procedure during a surgery to ablate a tissue mass such as a uterine fibroid tumor. In particular, such control is achieved in a multiple stylet ablation system by the robotically controlled or assisted control platform.

One embodiment for the combination of the ablation system deployed via a robotically controlled or assisted system may include a minimally invasive telesurgical system, or robotically controlled surgical system which includes a control station, or surgeon's console. The control station may include an image display or viewer where an image of a surgical site is displayed in use. In one variation, the images displayed on monitor may be displayed to the surgeon within image display or viewer. A support is provided on which an operator, typically a surgeon, can rest his or her forearms while gripping two master control devices, one in each hand. The master control devices are positioned in a space inwardly beyond the support. When using the control station, the surgeon typically sits in a chair in front of the control station, positions his or her eyes in front of the viewer and grips the master controls one in each hand while resting his or her forearms on the support.

The system further includes a surgical work station, or cart, which in use is positioned in close proximity to a patient requiring surgery and is then normally caused to remain stationary until a surgical procedure to be performed by means of the system has been completed. The cart typically carries at least three robotic arm assemblies. Each of the arm assemblies is arranged to hold a robotically controlled surgical instrument. Each robotic arm assembly is normally operatively connected to one of the master controls. Thus, the movement of the robotic arm assemblies is controlled by manipulation of the master controls. When a surgical procedure is to be performed, the cart carrying the robotic arms is wheeled to the patient and is normally maintained in a stationary position relative to, and in close proximity to, the patient, during the surgical procedure.

The instruments on the robotic arm assemblies may have a corresponding instrument for use with the ablation system. In one variation, a first robotic arm assembly may have an ultrasound probe coupled, a second robotic arm assembly may have an ablation probe with deployable stylets, and a third robotic arm assembly may have a laparoscope or endoscope which may provide a visual image of the surgical field during surgery within the patient body. The visual images received from the laparoscope or endoscope as well as the ultrasound images received from ultrasound probe may be displayed within the display area of the viewer while parameters from the ablation probe may also be displayed within the viewer.

Because of the computer control of the robotic arm assemblies, the positioning of the arm assemblies and their respective instruments in space and relative to the patient may be automatically tracked at all times by the station thus providing a method for navigating the instrument position. Furthermore, the positioning of the instruments relative to one another and to the tissue region of interest may be displayed by the station during use.

In one example of use, the surgical work station may be positioned in proximity to the patient positioned upon the surgical platform. The control station may be positioned at a distance remote from the surgical work station while the two remain in communication with one another. The ablation control system may also remain with the control station remote from the patient. The surgeon may be positioned in front of the control station to control the movement of the first, second and third robotic arm assemblies to control and manipulate the respective ultrasound probe, ablation probe, and laparoscope or endoscope via the control station to visual and ablate tissue regions of interest, e.g., uterine fibroids, within the patient. Additional monitors may also be optionally incorporated with the control station to display any number of additional information of the procedure and/or patient.

In one embodiment, the ablation system may generally comprise a surgical workstation having one or more robotic arm assemblies and configured to be in proximity to a surgical region of interest and a control station in communication with the surgical workstation and configured to control a positioning of each of the robotic arm assemblies. The system may also include an ultrasound instrument operably coupled to a first robotic arm assembly, an ablation instrument having a plurality of deployable stylets reconfigurable from a low-profile configuration to a deployed configuration, wherein the ablation instrument is operably coupled to a second robotic arm assembly, and an imaging instrument operably coupled to a third robotic arm assembly. In other variations, each instrument may be configured for use by a respective robotic arm assembly having an articulatable grip.

In one method of use, the method of ablating tissue may generally comprise positioning an ultrasound instrument, ablation instrument, and imaging instrument in proximity to a surgical region of interest, wherein the ultrasound instrument is operably coupled to a first robotic arm assembly, the ablation instrument is operably coupled to a second robotic arm assembly, and the imaging instrument is operably coupled to a third robotic arm assembly. The method may further include imaging the surgical region of interest via the ultrasound instrument while controlling a position of the ultrasound instrument via the first robotic arm assembly and displaying an image of the ultrasound instrument and the surgical region of interest. Furthermore, the method may include ablating the surgical region of interest via the ablation instrument while controlling a position of the ultrasound instrument via the second robotic arm assembly. In other variations, each instrument may be configured for use by a respective robotic arm assembly having an articulatable grip.

### BRIEF DESCRIPTION OF THE DRAWINGS

A full understanding of the invention can be gained from the following description of the preferred embodiments when read in conjunction with the accompanying drawings in which:

FIG. 1A illustrates a perspective view of an ablation device which may be used with a robotically controlled or assisted system.

FIG. 1B illustrates a schematic view of an ablation system incorporating computer controls which may be used with a robotically controlled or assisted system.

FIG. 2 illustrates a schematic view of a variation of the ablation system which may be used with a robotically controlled or assisted system.

FIG. 3 illustrates a schematic view of another variation of the ablation system having imaging data from two different image sources which may be used with a robotically controlled or assisted system.

FIG. 4 illustrates a perspective view of an operator control station and a surgical work station, or cart, of a telesurgical system having multiple robotically controlled arms for use with the ablation system.

FIGS. 5A and 5B illustrate perspective views of an ablation instrument which is controllable via the telesurgical system.

FIG. 6A illustrates a perspective view of an ultrasound instrument which is controllable via the telesurgical system.

FIG. 6B illustrates a perspective view of an imaging camera instrument which is controllable via the telesurgical system.

FIG. 7 illustrates a schematic view of the ablation system used with the telesurgical system for performing a procedure upon a patient.

### DETAILED DESCRIPTION OF THE INVENTION

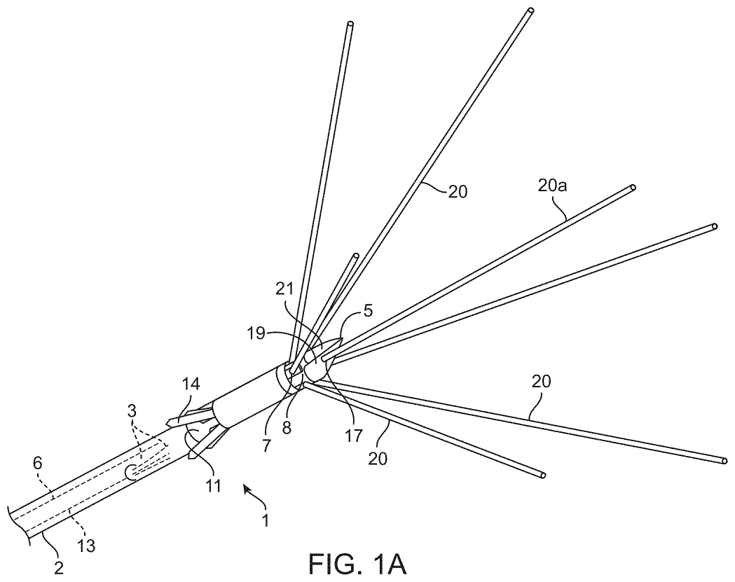

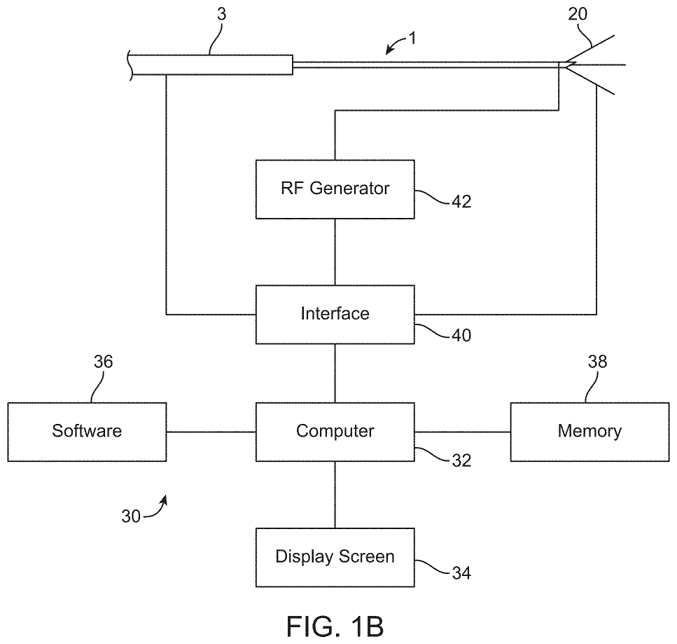

FIG. 1A is a perspective view of a multiple antennae or stylet ablation trocar instrument 1 which generally comprises a cannula 2 which houses a plurality of stylets 20 and, optionally, a plurality of anchors 14. A trocar point 5 is provided at the distal end of cannula 2. At least one conductor 6 is provided within the cannula 2 and is electrically coupled to the stylets 20 and trocar point 4 and accordingly provides RF energy to stylets 20 and trocar point 5. The stylets 20 and trocar point 5 may be electrically coupled to each other and electrically isolated from other exposed portions of the ablation instrument 1. The stylets 20 and trocar point 5 are at the distal end of ablation instrument 1 and each of the stylets may be made of thin wire-like tubular members and during the procedure is initially housed entirely within the cannula 2.

The stylets 20 are deployed for ablation by being advanced in the forward direction toward the distal end of ablation instrument 1 out from ablation instrument 1 through openings 7. As the stylets 20 are advanced through openings 7, they bear against deflection surfaces 8 which are defined in the metal body which defines trocar point 5 at the distal end of the cannula 2.

During use, the trocar point 5 may be used to initially pierce the tissue of the fibroid tumor. Optionally, a plurality of anchors 14, also housed within the ablation instrument 1, may be deployed rearwardly toward the proximal end of ablation instrument 1. During deployment, the anchors 14 may be deflected by the deflection surface 11 to move into the positions illustrated in FIG. 1. After deployment, the anchors 14 may optionally be used to prevent rearward movement of trocar point 5 during deployment of stylets 20.

The stylets 20 may be deployed through the use of a slideably mounted member 13 housed within the cannula 2 and coupled to the telesurgical system at its proximal end (as described in further detail herein). The anchors 14 are also deployed through the use of a slideably mounted operator member (not illustrated) housed within the cannula 2 and also coupled to the telesurgical system at its proximal end. The distal end of operator member 13 is coupled to stylets 3 which may thus be advanced an identical distance in unison.

An exemplary system is illustrated in FIG. 1B where the system 30 comprises a computer 32 which may be any control device, such as a microprocessor, personal computer or a more powerful or less powerful computer with a typical personal computer-type operating system. The computer 32 may include a display screen 34 which may be incorporated into the telesurgical system (as described in further detail herein) and may also optionally be a touchscreen to provide a second means of navigation.

The personal computer 32 may also incorporate software 36 which may be of any type for use on any suitable computing device, and which may be easily written by a programmer of ordinary skill in the art who is informed by this specification. The software is responsive to produce images illustrated in the drawings and stored in a memory 38 of computer 32. The software performs the navigation functions described above, being responsive to touchscreen entry and/or scroll and select buttons 23 and 25 on the ablation instrument 1.

The computer 32 communicates with ablation instrument 1 through an interface board 40 which is coupled to the telesurgical system Likewise, in response to operation by touching on display screen 34, the computer 32 may cause the RF generator 42 to apply power to the trocar point for ablation. In response thereto, thermocouples on stylets 20 will generate temperature indicating signals which are coupled through suitable interface electronics to computer 32, allowing the computer to control application of RF generator by RF generator 42, to display temperature information, operate alarms, to terminate the application of RF energy, and to perform any other design controls in response thereto, for example as described above.

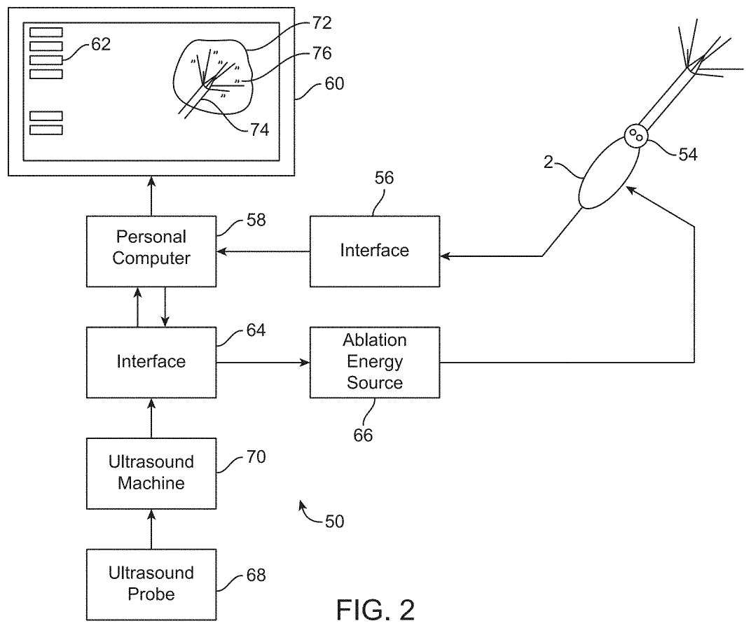

Temperature signals and control information are coupled to a computer interface 56 which sends this information to personal computer 58 which may drive a computer display 60 which includes a navigation menu 62 of the type described herein. The personal computer 58 through interface board 64 may control the ablation energy source 66 while an ultrasound probe 68 coupled to an ultrasound machine 70 may simultaneously provide ultrasound image information to interface 64 which in turn provides this information to personal computer 58 for display on computer display 60, as shown in the schematic view of FIG. 2.

The surgeon may concentrate on a single monitor displaying both ultrasound, and device performance information and a means for control of the system. More particularly, computer display 60 displays, for example, a fibroid 72 being operated on, an image 74 of ablation probe 54 and an image 76 of temperature data. The positioning of the images 74 and 76 may be done by the computer using a pattern matching or other strategy.

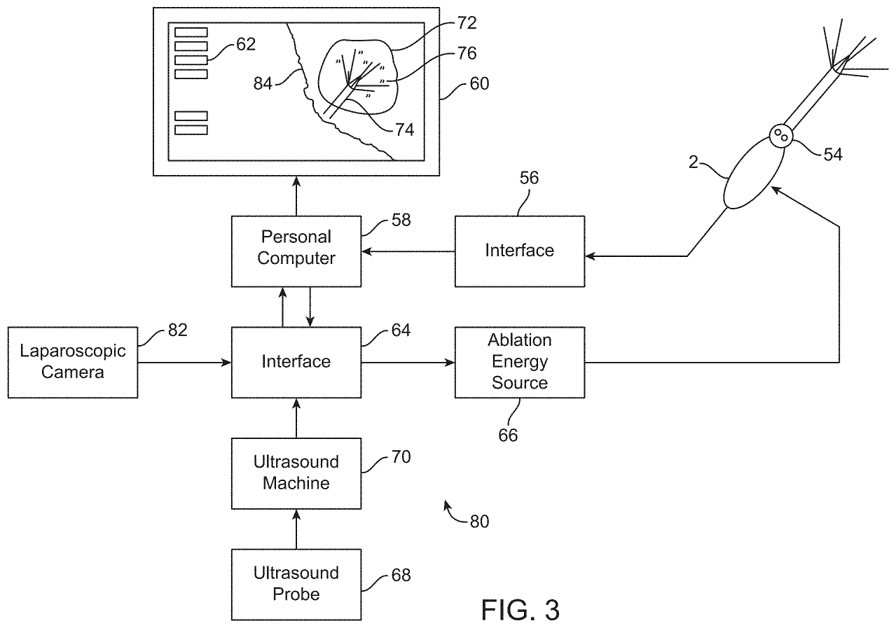

Another embodiment of the ablation system is illustrated in FIG. 3 which includes the addition and integration of an image from a laparoscope. A laparoscopic camera 82 may be coupled to interface 64 and camera 82 may be positioned by the surgeon to produce an image of the outside of the uterus resulting in display of an image 84 of the uterus on computer display 60 superimposed over the image 72 of the fibroid obtained using ultrasound. It is noted that images 72 and 84 are positioned in the same manner as the fibroid and the uterus are positioned in the patient, thus giving a more complete picture of the state of the surgery.

During use, when deploying the stylets 20 from the ablation device 16, a deployment length of the stylets 20 may be adjusted from any length of a partially extended configuration to a fully extended configuration. Depending upon the length of the deployed stylets 20 from the ablation device 16, the size of the ablation zone surrounding the stylets 20 will vary accordingly as well. Hence, the user may adjust the size of the ablation zone to match or correlate with the size of, e.g., a fibroid, as well as to minimize ablation of the tissue region surrounding the treated region.

To facilitate sizing of the treatment region, a visual representation of the ablation zone may be provided to the user so that the user may quickly confirm not only that the positioning of the ablation device 16 relative to the treatment area is sufficient but also that the deployment length of the stylets 20 is suitable for creating an ablation zone of sufficient size. Hence, a dynamic imaging system which automatically generates a visual representation of the ablation zone, based on specified parameters, may be provided.

Additional details of the ablation system are described in further detail in the following references, each of which is incorporated herein by reference in its entirety and for any purpose: U.S. Pat. Nos. 6,840,935; 7,678,106; 8,080,009; 8,241,276; 8,251,991; 8,512,330; 8,512,333; 9,510,898; 9,662,166; 9,861,426; U.S. Pat. Pub. Nos. 2008/0045940; 2012/0245575; 2012/0245576; 2015/0190206; 2016/0095537; 2017/0333116; 2018/0125566.

As discussed above, the ablation system may be deployed via any number of robotically controlled or assisted systems such as the da Vinci® surgical platform (Intuitive Surgical, Inc., Sunnyvale, Calif.). Examples of this robotic surgical platform are described, for example, in U.S. Pat. No. 6,312,435 which is incorporated herein by reference in its entirety and for any purpose.

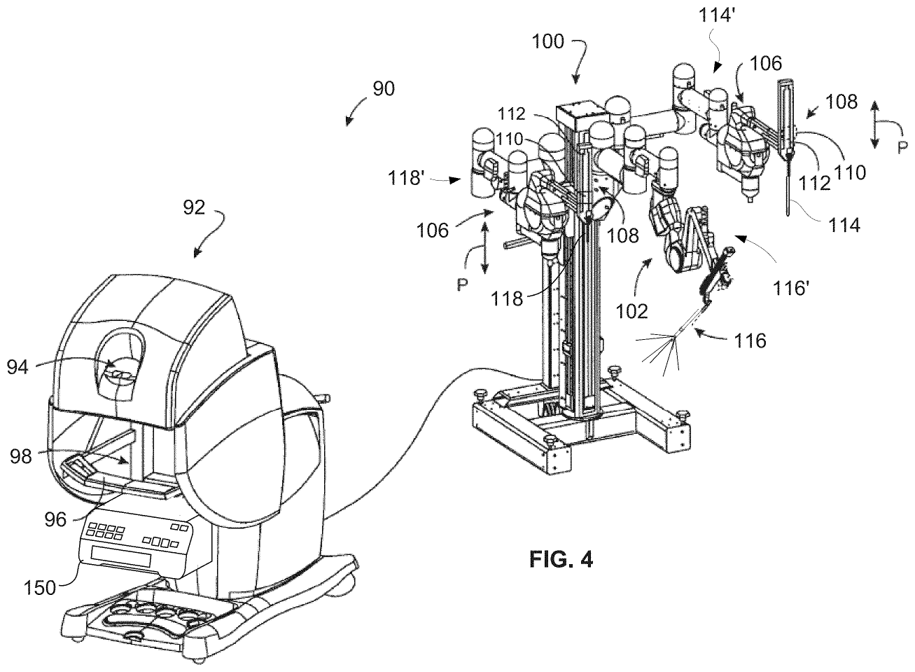

One embodiment for the combination of the ablation system deployed via a robotically controlled or assisted system is shown in the perspective view of FIG. 4 which illustrates a minimally invasive telesurgical system 90, or robotically controlled surgical system which includes a control station 92, or surgeon's console. The control station 92 may include an image display or viewer 94 where an image of a surgical site is displayed in use. In one variation, the images displayed on monitor 60, as described above, may be displayed to the surgeon within image display or viewer 94. A support 96 is provided on which an operator, typically a surgeon, can rest his or her forearms while gripping two master control devices, one in each hand. The master control devices are positioned in a space 98 inwardly beyond the support 96. When using the control station 92, the surgeon typically sits in a chair in front of the control station 92, positions his or her eyes in front of the viewer 94 and grips the master controls one in each hand while resting his or her forearms on the support 96.

The system 90 further includes a surgical work station 100, or cart, which in use is positioned in close proximity to a patient requiring surgery and is then normally caused to remain stationary until a surgical procedure to be performed by means of the system 90 has been completed. The cart 100 may have wheels or castors to render it mobile. The station 92 is typically positioned remote from the cart 100 and can be separated from the cart 100 by a great distance, even miles away, but will typically be used within an operating room with the cart 100.

The cart 100 typically carries at least three robotic arm assemblies. Each of the arm assemblies 102, 106, 108, respectively, is arranged to hold a robotically controlled surgical instrument 108. Each robotic arm assembly 106 is normally operatively connected to one of the master controls. Thus, the movement of the robotic arm assemblies 106 is controlled by manipulation of the master controls. When a surgical procedure is to be performed, the cart 100 carrying the robotic arms 102, 106, 106 is wheeled to the patient and is normally maintained in a stationary position relative to, and in close proximity to, the patient, during the surgical procedure.

The instruments on the robotic arm assemblies 106 may have a corresponding instrument for use with the ablation system. In one variation, a first robotic arm assembly 114′ may have an ultrasound probe 114 coupled, a second robotic arm assembly 116′ may have an ablation probe 116 with deployable stylets 20, and a third robotic arm assembly 118′ may have a laparoscope or endoscope 118 which may provide a visual image of the surgical field during surgery within the patient body. The visual images received from the laparoscope or endoscope 118 as well as the ultrasound images received from ultrasound probe 114 may be displayed within the display area of the viewer 94 while parameters from the ablation probe 116 may also be displayed within the viewer 94.

While each of the instruments may be integrated directly with a respective robotic arm assembly, other variations of the system may have the robotic arm assemblies attached or otherwise gripping a respective instrument. For instance, the robotic arm assemblies 114′, 116′, 118′ may be configured with an articulatable gripping mechanism which may be used to secure each instrument 114, 116, 118.

Because of the computer control of the robotic arm assemblies, the positioning of the arm assemblies and their respective instruments in space and relative to the patient may be automatically tracked at all times by the station 92 thus providing a method for navigating the instrument position. Furthermore, the positioning of the instruments relative to one another and to the tissue region of interest may be displayed by the station 92 during use.

Alternatively, the instruments may be positioned via an instrument navigation system separate from the robotic arm assemblies. For instance, a position and orientation of each of the individual instruments may be tracked in space relative to one another via an electromagnetic field generator such as one described in further detail in U.S. Pat. Pub. No. 2016/0095537, which has been incorporated herein above by reference in its entirety. Such an electromagnetic field generator may be in communication with the station 92 to provide instrument position and orientation relative to one another as well as relative to the patient body and surgical region of interest for display to the physician.

It will be appreciated that the instruments 108 have elongate shafts to permit the end effectors to be inserted through entry ports in a patient's body so as to access the internal surgical site. Movement of the end effectors relative to the ends of the shafts of the instruments 108 is also controlled by the master controls.

While the robotic arm assemblies may be controlled by the control station 92, an additional controller 150 may be incorporated with the control station 92 to separately control and/or monitor the functions and operations of the ablation system itself, including the ablation probe 116, ultrasound probe 114, and laparoscopic instrument 118. The controller 150 may combine the functionality of the ablation system into a single control unit 150 or the functionality may be integrated into the control station 92 as a singular unit. Additional details of the control 150 and the ablation system are described in U.S. patent application Ser. No. 16/186,215 filed Nov. 9, 2018, which is incorporated herein by reference in its entirety and for any purpose.

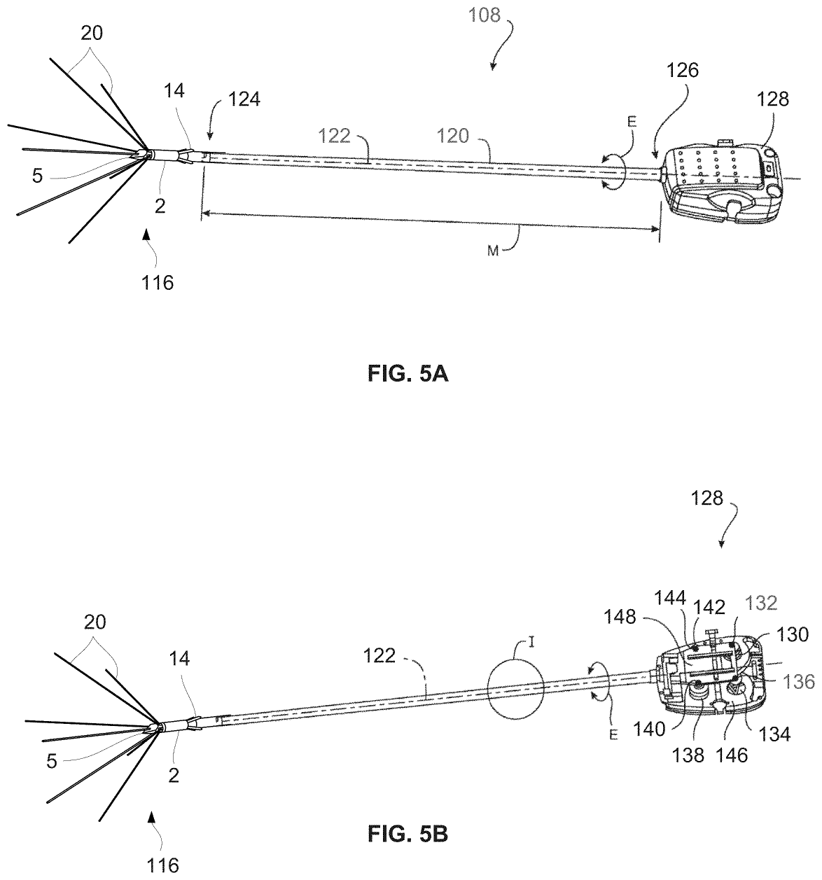

Referring to FIG. 5A, the surgical instrument 108 will now be described in greater detail. The surgical instrument 108 includes an elongate shaft 120 having opposed ends 124 and 126 and a length M, which may be varied depending upon the application, e.g., between about 250 mm and about 560 mm, or a length of about 400 mm. Furthermore, the shaft 108 may have an outer cross-sectional dimension of less than, e.g., between about 3 mm and about 92 mm. The ablation probe 116 is located at the end 124 of the shaft 120 with the stylets 20 and anchors 14 shown in their deployed configuration. A housing 128, arranged releasably to couple the instrument 108 to one of the robotic arm assemblies 106 is located at the other end 126 of the shaft 120. The ablation probe 116 is typically releasably mountable on a carriage which can be driven to translate along a linear guide formation 112 of the arm 106 in the direction of arrows P.

The surgical instrument 108 typically has four transmission members 130, 134, 138, and 142 which are typically in the form of drums or spools. The spools 130, 134, 138, 142 are secured on shafts 132, 136, 140, 144, respectively, which may extend through a base 146 of the housing 128. Ends of the shafts 132, 136, 140, 144 are rotatably held by and between a mounting plate 148 and the base 146 and opposed ends of the shafts 132, 136, 140, 144 extend through the base 146, to an opposed side of the base, hidden from view in FIG. 5B. At the opposed side, each shaft 132, 136, 140, 144 carries an engaging member (not shown) on its opposed end. Each engaging member is arranged releasably to couple with a complementary engaging member (not shown) rotatably mounted on the carriage. The engaging members on the carriage are operatively connected to actuators (not shown), e.g., electric motors, or the like, to cause selective angular displacement of each engaging member on the carriage in response to actuation of its associated actuator. Thus, selective actuation of the actuators is transmitted through the engaging members on the carriage, to the engaging members on the opposed ends of the shafts 132, 136, 140, 144 to cause selective angular displacement of the spools 130, 134, 138, 142. Selective angular displacement of the spools 130, 134, 138, 142 causes selective actuation of the elongate actuation elements, which in turn causes selective angular displacement of the shaft 120 and ablation probe 116, and rotation E of the shaft 120 about its longitudinal axis 122.

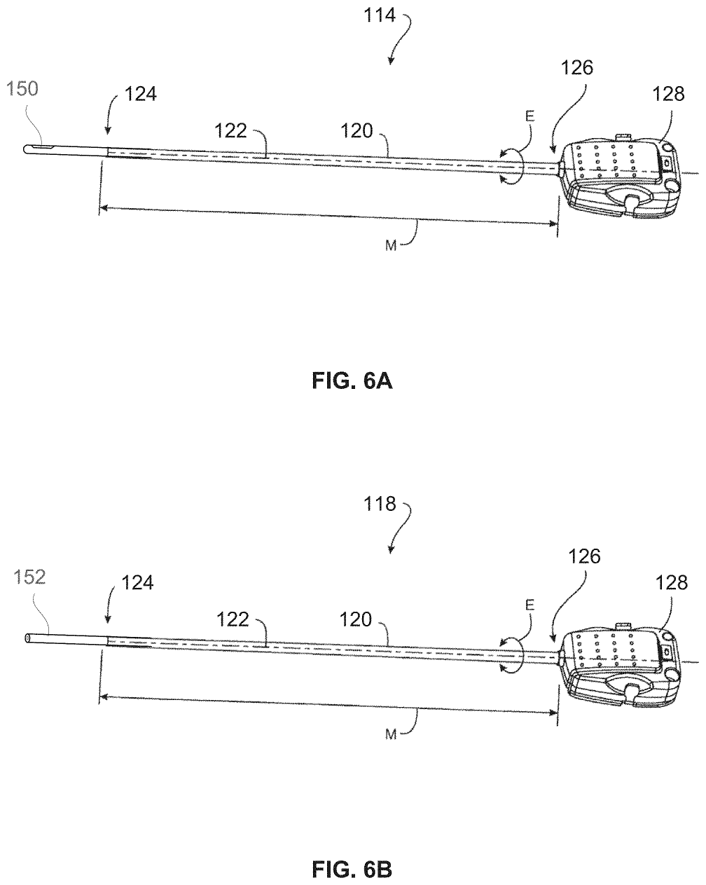

FIG. 6A shows a perspective view of the ultrasound probe 114 having an ultrasound transducer 150 mounted at a distal end of the shaft 120. Similarly, FIG. 6B shows a perspective view of the laparoscope or endoscope 118 having an imager 152 mounted at a distal end of the shaft 120.

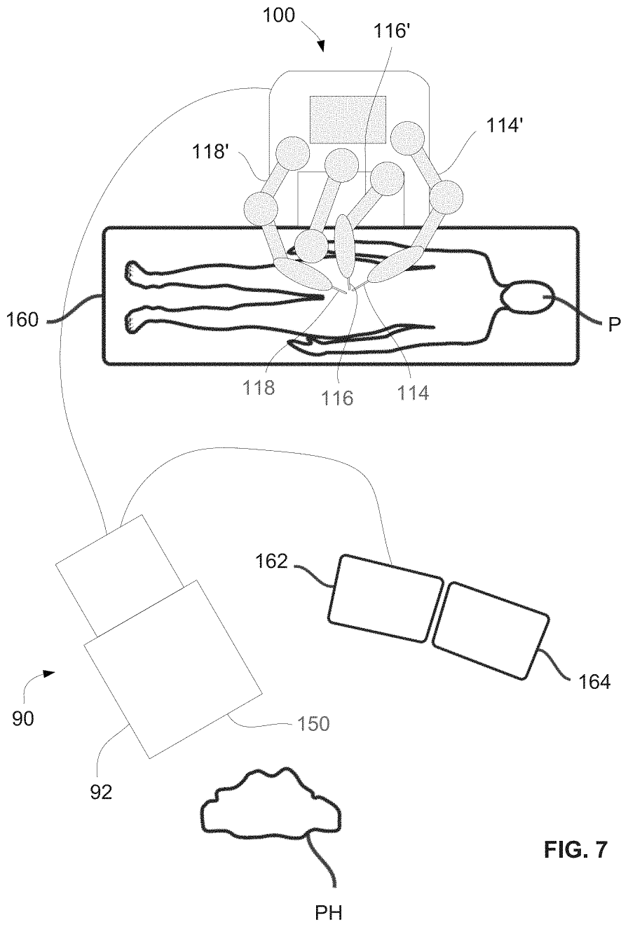

An example of how the robotically controlled telesurgical system 90 may be positioned for use is shown in the schematic illustration of FIG. 7. As shown, the surgical work station 100 may be positioned in proximity to the patient P positioned upon the surgical platform 160. The control station 92 may be positioned at a distance remote from the surgical work station 100 while the two remain in communication with one another. The ablation control system 150 may also remain with the control station 92 remote from the patient. The surgeon PH may be positioned in front of the control station 92 to control the movement of the first, second and third robotic arm assemblies 114′, 116′, 118′ to control and manipulate the respective ultrasound probe 114, ablation probe 116, and laparoscope or endoscope 118 via the control station 92 to visual and ablate tissue regions of interest, e.g., uterine fibroids, within the patient P. Additional monitors 162, 164 may also be optionally incorporated with the control station 92 to display any number of additional information of the procedure and/or patient P.

It will be appreciated by those skilled in the art that changes can be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. An ablation system, comprising:
a surgical workstation having one or more robotic arm assemblies and configured to be in proximity to a surgical region of interest;
a control station in communication with the surgical workstation and configured to control a positioning of each of the robotic arm assemblies;
an ultrasound instrument configured for use by a first robotic arm assembly;
an ablation instrument having a plurality of deployable stylets reconfigurable from a low-profile configuration to a deployed configuration, wherein the ablation instrument is configured for use by a second robotic arm assembly; and
an imaging instrument configured for use by a third robotic arm assembly.

2. The system of claim 1 wherein each of the one or more robotic arm assemblies is configured to articulate a position of an instrument operably coupled.

3. The system of claim 1 wherein the surgical workstation is movable into proximity to the surgical region of interest.

4. The system of claim 1 wherein the control station comprises a visual display.

5. The system of claim 1 wherein the control station further comprises a controller in communication with the ultrasound instrument, ablation instrument, and imaging instrument.

6. The system of claim 1 wherein the control station is configured to track a position and/or orientation of one or more of the instruments relative to one another and the surgical region of interest.

7. The system of claim 1 further comprising a field generator configured to track a position and/or orientation of one or more of the instruments relative to one another and the surgical region of interest.

8. A method of ablating tissue, comprising:
positioning an ultrasound instrument, ablation instrument, and imaging instrument in proximity to a surgical region of interest, wherein the ultrasound instrument is configured for use by a first robotic arm assembly, the ablation instrument is operably configured for use by a second robotic arm assembly, and the imaging instrument is configured for use by a third robotic arm assembly;
imaging the surgical region of interest via the ultrasound instrument while controlling a position of the ultrasound instrument via the first robotic arm assembly;
displaying an image of the ultrasound instrument and the surgical region of interest; and
ablating the surgical region of interest via the ablation instrument while controlling a position of the ultrasound instrument via the second robotic arm assembly.

9. The method of claim 8 wherein the first, second, and third robotic arm assemblies are coupled to a surgical workstation positioned in proximity to the surgical region of interest.

10. The method of claim 8 wherein the first, second, and third robotic arm assemblies are controlled via a control station located remotely from the surgical region of interest.

11. The method of claim 8 wherein displaying an image of the ultrasound instrument comprises displaying the image upon a display located within a surgical workstation.

12. The method of claim 8 wherein ablating the surgical region of interest comprises deploying a plurality of stylets into a tissue region via the second robotic arm assembly.

13. The method of claim 8 further comprising tracking a location of each of the robotic arm assemblies.
